(19) United States
(12) Patent Application Publication
(10) Pub. No.: US 2024/0387042 A1
(43) Pub. Date: Nov. 21, 2024

(54) ARTIFICIAL INTELLIGENCE SYSTEM FOR SENSING AND REHABILITATION

(71) Applicants: Szu-Wei Sun, Taoyuan City (TW); Yu-Han Sun, Taoyuan City (TW)

(72) Inventors: Szu-Wei Sun, Taoyuan City (TW); Yu-Han Sun, Taoyuan City (TW)

(21) Appl. No.: 18/318,838

(22) Filed: May 17, 2023

(51) Int. Cl. G16H 50/20; G06T 7/00; G06V 40/10; G16H 20/30

(52) U.S. Cl. CPC G16H 50/20; G06T 7/0012; G06V 40/10; G16H 20/30; G06T 2207/30196

(57) ABSTRACT

An AI system includes a control device including storage, learning unit and display units, and a CPU electrically connected to the storage, the learning and the display units in which the storage unit stores diagnosis data; the learning unit includes a learning machine and the learning unit inputs the diagnosis data into the learning machine; and the learning machine uses a machine learning algorithm to compare data and establish a visualized determination model; an auxiliary diagnosis device electrically connected to the CPU in which the auxiliary diagnosis device includes mechanical arms for moving or arranging a fixing position of a body part of a participant and rehabilitating the participant; and an image sensing device electrically connected to the CPU for generating a first monitor signal and sending same to the CPU to process. The CPU compares the first monitor signal with the determination model.

### FIELD OF THE INVENTION

The invention relates to rehabilitation systems and more particularly to an artificial intelligence (AI) system for sensing and rehabilitation.

### BACKGROUND OF THE INVENTION

In the conventional field of physical therapy, a physical therapist generally starts with a patient's chief complaint, and then uses his (or her) own experience to evaluate and judge individually, so as to treat or rehabilitate the patient. However, the biggest dilemma for the physical therapists is that the time provided to each patient is fixed. If the evaluation takes too long time, it will decrease the rehabilitation treatment time, resulting in insufficient rehabilitation treatment time.

And evaluation and judgment depend on the personal experience of the physical therapist. On the measurement tools, traditional tools are also used to monitor the patient. For example, the range of motion of the patient's knee flexion is measured with a goniometer. As a result, there is a lack of standardization for the evaluation and judgment or rehabilitation treatment methods and accuracy thereof is very low, especially the body shape of each patient is different, and so are the anatomy positions of their bodies. Thus, it is more likely to have mistakes in judgment. In addition, the largest difficulty is that the physical therapist only has two hands during the treatment process. If the patient is not allowed to apply force, many assessment and treatment actions actually require more arms to assist (e.g., the assistance of a nurse).

Thus, the need for improvement still exists.

### SUMMARY OF THE INVENTION

It is therefore one object of the invention to provide an artificial intelligence system for sensing and rehabilitation comprising a control device including a central processing unit (CPU), a storage unit, a learning unit, and a display unit wherein the storage unit, the learning unit, and the display unit are electrically connected to the CPU respectively; the storage unit stores a plurality of diagnosis data; the learning unit includes a learning machine and the learning unit inputs the diagnosis data into the learning machine; and the learning machine uses a machine learning algorithm to compare data and establish a visualized determination model; an auxiliary diagnosis device electrically connected to the CPU and controlled by the control device wherein the auxiliary diagnosis device includes a plurality of mechanical arms controlled by the control device to move or arrange a fixing position of a body part of a participant and rehabilitate the participant; and an image sensing device electrically connected to the CPU and controlled by the control device wherein the image sensing device is configured to generate a first monitor signal and send same to the CPU to process, and the CPU compares the first monitor signal with the determination model.

Preferably, the auxiliary diagnosis device further comprises at least one physiological sensing device for generating a second monitor signal and sending same to the CPU to process wherein the CPU compares the second monitor signal with the determination model.

The above and other objects, features and advantages of the invention will become apparent from the following detailed description taken with the accompanying drawings

### BRIEF DESCRIPTION OF THE DRAWINGS

FIG. 1 is a block diagram of an AI system for sensing and rehabilitation according to the invention;

FIG. 2 is a flow chart of operating the AI system for sensing and rehabilitation according to the invention;

FIG. 3 is an environmental view showing the AI system being used to monitor de Quervain syndrome in which a physiological sensing device is attached to a mechanical arm which is in turn attached to an arm of a participant;

FIG. 4 is similar to FIG. 3 in which three physiological sensing devices are attached to three mechanical arms respectively; and

FIG. 5 is an environmental view showing the AI system being used to monitor ranges of motion (ROMs) of joints of a participant lying on a physical therapy bed.

### DETAILED DESCRIPTION OF THE INVENTION

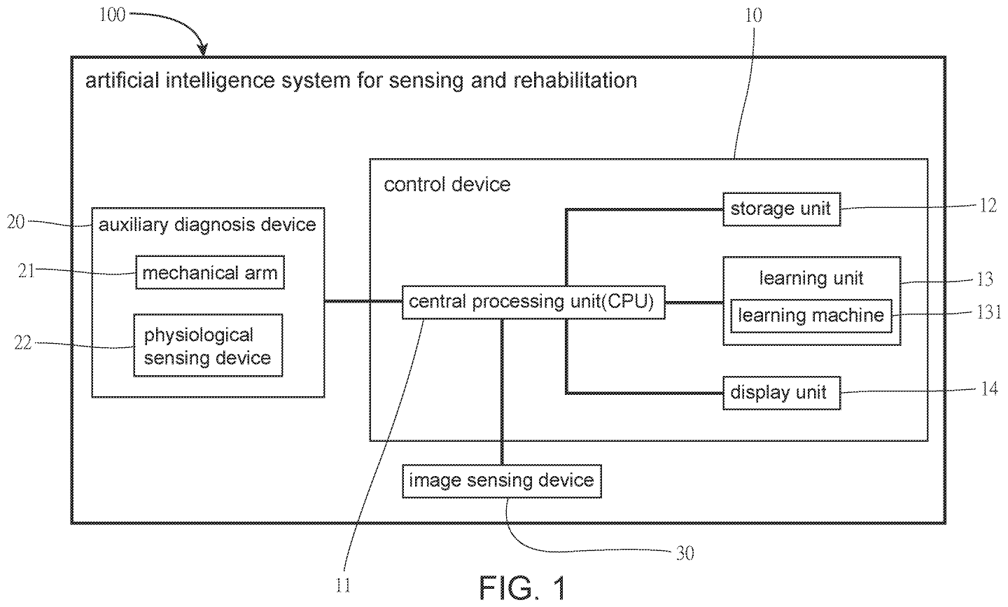

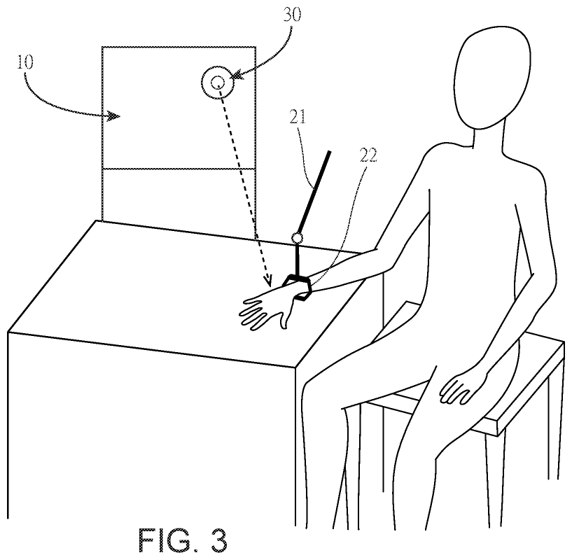

Referring to FIGS. 1 and 3, an AI system 100 for sensing and rehabilitation in accordance with the invention comprises the following components as discussed in detail below.

A control device 10 is provided. The control device 10 is implemented as a computer and includes a central processing unit (CPU) 11, a storage unit 12, a learning unit 13, and a display unit 14. The storage unit 12, the learning unit 13, and the display unit 14 are electrically connected to the CPU 11 respectively. The storage unit 12 stores a plurality of diagnosis data including correct positions of anatomy such as bones, muscles, nerves, blood vessels, tendons, ligaments and joints of the human bodies of a plurality of healthy persons, signs and symptoms of various illnesses, rehabilitation methods for the various illnesses, and arranged fixing positions. The learning unit 13 includes a learning machine 131 and the learning unit 13 inputs the diagnosis data into the learning machine 131. The learning machine 131 uses a machine learning algorithm to compare data and establish a visualized determination model. The display unit 14 is implemented as a computer monitor for displaying information including operating statuses, images, and the determination model which has been compared.

An auxiliary diagnosis device 20 is provided. The auxiliary diagnosis device 20 is electrically connected to the CPU 11 and controlled by the control device 10. The auxiliary diagnosis device 20 includes a plurality of mechanical arms 21 and at least one physiological sensing device 22. The mechanical arms 21 are controlled by the control device 10 to move or arrange a fixing position of a body part of a participant and rehabilitate the participant. The physiological sensing device 22 is independently provided or attached to an end of the mechanical arm 21. As shown in FIG. 3 specifically, an infrared (IR) thermometer is integrated into the physiological sensing device 22. The physiological sensing device 22 is used to detect physical data (e.g., pulse, breath, etc.) of the participant; detect whether there is inflammation on the muscles and the bones of a target part of the participant and whether there is inflammation on the blood vessels and the system of nerves of the participant; and generate a second monitor signal and send same to the CPU 11 to process.

An image sensing device 30 is electrically connected to the CPU 11. The image sensing device 30 is provided in the control device 10 or externally of the control device 10 and is controlled by the control device 10. The image sensing device 30 is used to detect physical data of a target part of the participant (e.g., the posture or the part not felt well); detect whether there is inflammation on the nerves, the tendons, and the ligaments of the participant; and generate a first monitor signal and send same to the CPU 11 for processing.

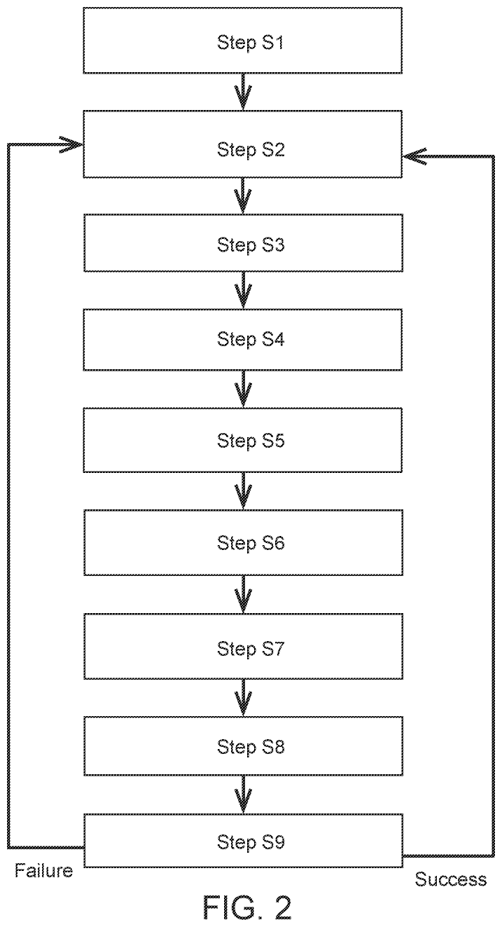

Referring to FIG. 2, a flow chart of operating the AI system for sensing and rehabilitation of the invention is illustrated. The operation comprises the following steps:
Step S1: entering diagnosis data into the storage unit 12 to store as a diagnosis database;
Step S2: entering the diagnosis database into the learning machine 131 of the learning unit 13 so that the learning machine 131 may use a machine learning algorithm to compare data, analyze the data, and establish a visualized determination model;
Step S3: sending a target part of the participant to the control device 10;
Step S4: activating the control device 10 to drive the image sensing device 30 to detect the position of the target part of the participant and generating corresponding information or detecting whether there is inflammation on the nerves, the tendons, and the ligaments of the participant based on needs; and generating a first monitor signal;
Step S5: activating the control device 10 to drive the mechanical arm 21 to move or arrange a fixing position of a body part of the participant; activating the physiological sensing device 22 to perform physiological checks or detect whether there is inflammation on the muscles and the bones of a target part of the participant and whether there is inflammation on the blood vessels and the system of nerves of the participant; and generating a second monitor signal in which the image sensing device 30 may monitor and collect data in real time;
Step S6: sending the first and second monitor signals to the CPU 11 for processing;
Step S7: instructing the CPU 11 to compare the first and second monitor signals with the determination model and generate a rehabilitation program based on a result of the comparison;
Step S8: activating the mechanical arm 21 to rehabilitate the participant based on the rehabilitation program; and
Step S9: finishing the rehabilitation. The CPU 11 will report the rehabilitation result to the learning unit 13 (i.e., returning to step S2) without considering whether the conditions of the participant have been improved or not (i.e., success or failure). If the rehabilitation result is failure, the CPU 11 generates a new rehabilitation program to repeat step S8 until the rehabilitation result is success. Also, the learning unit 13 updates the determination model and relearns to increase accuracy of the determination model.

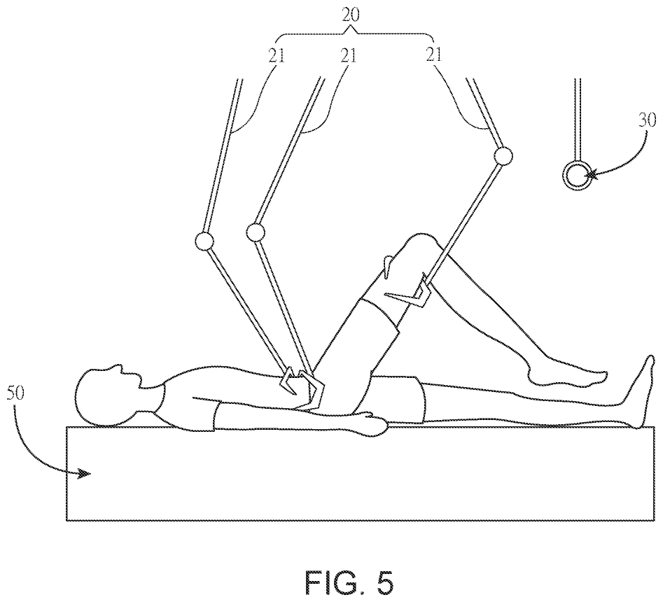

Referring to FIG. 5 in conjunction with FIG. 1, the AI system 100 is used to monitor ROMs of joints of a participant in which the hip flexion is taken as an example. First, the display unit 14 instructs the participant to lie on a physical therapy bed 50. The image sensing device 30 determines whether the posture of the participant is correct. Two mechanical arms 21 are used to fix the hip and the trunk of the participant. The image sensing device 30 takes greater trochanter of femur as a pivot, trunk as a fixing end, and lateral epicondyle of femur as a moving end. The third mechanical arm 21 moves the posterior surface of a distal end of femur of the participant to flex the hip joint. The image sensing device 30 monitors the passive range of motion (PROM) of the joint of the participant to generate a first monitor signal and send it to the CPU 11. Finally, the learning unit 13 compares the first monitor signal with the determination model and determines whether the ROM of the joint is in the normal range based on a result of the comparison.

Manual muscle testing (MMT) is conducted. Specifically, quadriceps is taken as an example. The participant follows the instructions shown on the display unit 14 to sit on the physical therapy bed 50. The image sensing device 30 determines whether the posture of the participant is correct. Two mechanical arms 21 are used to fix the hip and the trunk of the participant. The third mechanical arm 21 applies force to the hip extension at a proximate end of the knee joint of the participant. In turn, the participant follows the instructions shown on the display unit 14 to resist the force exerted by the third mechanical arm 21 to the proximate end of the knee joint of the participant. The physiological sensing device 22 senses the force to generate a second monitor signal which is sent to the CPU 11. Finally, the learning unit 13 compares the second monitor signal with the determination model and determines the muscle strength of the participant based on the strength of the resistance.

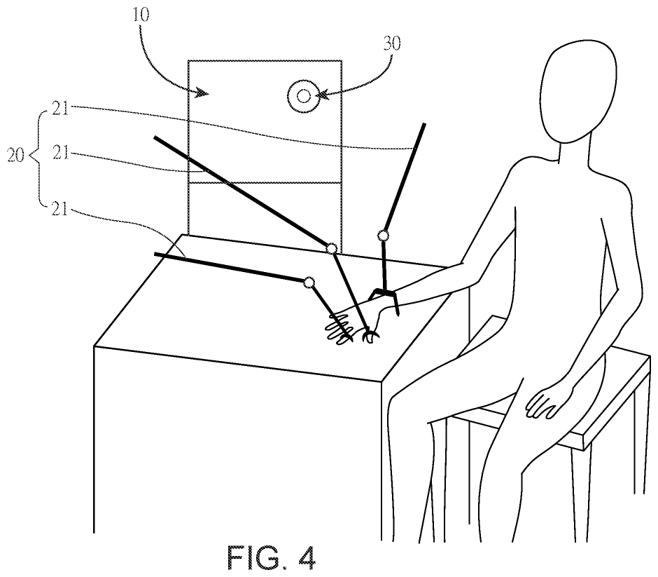

Referring to FIG. 4 in conjunction with FIG. 1, the AI system 100 is used to monitor de Quervain syndrome. When the thumb of the participant cannot exert force or the hand of the participant feels pain when exerting force, the control device 10 initially determines that the participant has de Quervain syndrome. And in turn, the control device 10 activates both the image sensing device 30 to monitor the participant's appearance and the physiological sensing device 22 to detect physical data (e.g., whether an IR sensor has sensed that there is temperature rise at inflammation of a body part of the participant). The image sensing device 30 sends the first monitor signal to the CPU 11 and the physiological sensing device 22 sends the second monitor signal to the CPU 11 respectively. It is determined that the participant has de Quervain syndrome if a reaction is positive. The control device 10 activates the mechanical arms 21 to measure ROMs and conduct MMT if the reaction is negative. At the same time, the control device 10 activates the physiological sensing device 22 to sense and sends the first and second monitor signals to the CPU 11. And in turn, the CPU 11 compares the first and second monitor signals with the determination model and generates a rehabilitation program based on a result of the comparison. Finally, the mechanical arms 21 are driven to rehabilitate the participant based on the rehabilitation program.

While above embodiment is directed to orthopedic physical therapy, the invention is equally applicable to neurological physical therapy, cardiopulmonary physical therapy, and pediatric physical therapy.

Moreover, the AI system 100 for sensing and rehabilitation can be implemented as an AI fitness coach. In short, the control device 10 issues an exercise prescription, the image sensing device 30 monitors accuracy and safety of the exercise prescription, and the auxiliary diagnosis device 20 helps a patient with guiding his (or her) exercise and adjusting his (or her) posture.

The invention has the following advantages and benefits in comparison with the conventional art: all evaluations and rehabilitation programs can be modularized as standardized rehabilitation methods. With the cooperation of the image sensing device 30, the mechanical arms 21, and the physiological sensing device 22, evaluation efficiency and accuracy of the rehabilitated part can be increased greatly, an optimum rehabilitation program can be quickly found by means of the current determination model without resorting to a physical therapist's experience, and the AI system for sensing and rehabilitation can be improved by means of every machine learning and updates so that a therapist can easy find causes of a disease by means of the AI system for sensing and rehabilitation.

While the invention has been described in terms of preferred embodiments, those skilled in the art will recognize that the invention can be practiced with modifications within the spirit and scope of the appended claims.

What is claimed is:

1. An artificial intelligence system for sensing and rehabilitation, comprising:
a control device including a central processing unit (CPU), a storage unit, a learning unit, and a display unit wherein the storage unit, the learning unit, and the display unit are electrically connected to the CPU respectively; the storage unit stores a plurality of diagnosis data; the learning unit includes a learning machine and the learning unit inputs the diagnosis data into the learning machine; and the learning machine uses a machine learning algorithm to compare data and establish a visualized determination model;
an auxiliary diagnosis device electrically connected to the CPU and controlled by the control device wherein the auxiliary diagnosis device includes a plurality of mechanical arms controlled by the control device to move or arrange a fixing position of a body part of a participant and rehabilitate the participant; and
an image sensing device electrically connected to the CPU and controlled by the control device wherein the image sensing device is configured to generate a first monitor signal and send it to the CPU to process, and the CPU compares the first monitor signal with the determination model.

2. The artificial intelligence system for sensing and rehabilitation of claim 1, wherein the auxiliary diagnosis device further comprises at least one physiological sensing device for generating a second monitor signal and sending same to the CPU to process wherein the CPU compares the second monitor signal with the determination model.

3. The artificial intelligence system for sensing and rehabilitation of claim 2, wherein the at least one physiological sensing device is independently provided or attached to one of the mechanical arms.

4. The artificial intelligence system for sensing and rehabilitation of claim 1, wherein the image sensing device is provided in the control device or externally of the control device.

5. The artificial intelligence system for sensing and rehabilitation of claim 1, wherein the diagnosis data comprises correct positions of anatomy including bones, muscles, nerves, blood vessels, tendons, ligaments, and joints of human bodies of a plurality of healthy persons; signs and symptoms of various illnesses; rehabilitation methods for the various illnesses; and arranged fixing positions.

6. The artificial intelligence system for sensing and rehabilitation of claim 1, wherein the display unit is configured to display information including operating statuses, images, and the determination model which has been compared.

7. The artificial intelligence system for sensing and rehabilitation of claim 1, further comprising a physical therapy bed configured to allow the participant to lie or sit thereon.

8. The artificial intelligence system for sensing and rehabilitation of claim 1, wherein the artificial intelligence system is configured to implement as an artificial intelligence fitness coach; and wherein the control device issues an exercise prescription, the image sensing device monitors accuracy and safety of the exercise prescription, and the auxiliary diagnosis device helps a patient with guiding his (or her) exercise and adjusting his (or her) posture.
